SEQUENCE LISTING (42 sequences)

SEQ ID NO: 1 - protein, 90 aa, unidentified
  VARIANT 7..90: note = Any of resides 7 - 90 may be missing
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGS

SEQ ID NO: 2 - protein, 30 aa, unidentified
  VARIANT 3..30: note = any of resides 3 - 30 may be missing
  VARIANT 2: note = wherein X=V, I, or A
  VARIANT 4: note = wherein X=V, I, or A
  VARIANT 6: note = wherein X=V, I, or A
  VARIANT 8: note = wherein X=V, I, or A
  VARIANT 10: note = wherein X=V, I, or A
  VARIANT 12: note = wherein X=V, I, or A
  VARIANT 14: note = wherein X=V, I, or A
  VARIANT 16: note = wherein X=V, I, or A
  VARIANT 18: note = wherein X=V, I, or A
  VARIANT 20: note = wherein X=V, I, or A
  VARIANT 22: note = wherein X=V, I, or A
  VARIANT 24: note = wherein X=V, I, or A
  VARIANT 26: note = wherein X=V, I, or A
  VARIANT 28: note = wherein X=V, I, or A
  VARIANT 30: note = wherein X=V, I, or A
GXGXGXGXGXGXGXGXGXGXGXGXGXGXGX

SEQ ID NO: 3 - protein, 4 aa, unidentified
GAAS

SEQ ID NO: 4 - protein, 15 aa, unidentified
  VARIANT 1..15: note = wherein one Serine residue may be missing
  VARIANT 1..15: note = wherein up to 2 Alanine residues may be missing
SSAAAAAAAAAAAAA

SEQ ID NO: 5-6 - skipped (2 entries)

SEQ ID NO: 7 - protein, 12 aa, unidentified
  VARIANT 2: note = wherein the serine residue may be missing
  VARIANT 8: note = wherein the serine residue may be missing
  VARIANT 4..12: note = wherein residues 4 - 12 may be missing
  VARIANT 4..6: note = wherein alanine residues may be missing
  VARIANT 10..12: note = wherein alanine residues may be missing
SSAAAASSAAAA

SEQ ID NO: 8 - protein, 6 aa, unidentified
GLGGLG

SEQ ID NO: 9 - protein, 6 aa, unidentified
  VARIANT 2: note = wherein X=L, I, V, P
  VARIANT 5: note = wherein X=L, I, V, P
GXGGXG

SEQ ID NO: 10 - protein, 15 aa, unidentified
  VARIANT 6..14: note = wherein residues 6 - 14 may be missing
  VARIANT 1..15: note = wherein the sequence can be repeated 'n' times
  VARIANT 5: note = wherein X=Y, V, S, A
  VARIANT 8: note = wherein X=Y, V, S, A
  VARIANT 11: note = wherein X=Y, V, S, A
  VARIANT 14: note = wherein X=Y, V, S, A
GPGGXGGXGGXGGXY

SEQ ID NO: 11 - protein, 5 aa, unidentified
  VARIANT 5: note = wherein the alanine residue may be repeated 'n' times
GRGGA

SEQ ID NO: 12 - protein, 13 aa, unidentified
  VARIANT 10: note = wherein the alanine residue may be missing
GAGAAAAAAAGGA

SEQ ID NO: 13 - protein, 8 aa, unidentified
  VARIANT 3: note = X=Q, Y, L, A, S, R
  VARIANT 5: note = X=Q, Y, L, A, S, R
  VARIANT 7: note = X=Q, Y, L, A, S, R
  VARIANT 8: note = X=Q, Y, L, A, S, R
GGXGXGXX

SEQ ID NO: 14 - protein, 16 aa, unidentified
TGSSGFGPYVNGGYSG

SEQ ID NO: 15 - protein, 8 aa, unidentified
YEYAWSSE

SEQ ID NO: 16 - protein, 7 aa, unidentified
SDFGTGS

SEQ ID NO: 17 - protein, 7 aa, unidentified
RRAGYDR

SEQ ID NO: 18 - protein, 8 aa, unidentified
EVIVIDDR

SEQ ID NO: 19 - protein, 17 aa, unidentified
TTHEDLDITIDGADGPI

SEQ ID NO: 20 - protein, 8 aa, unidentified
TISEELTI

SEQ ID NO: 21-22 - skipped (2 entries)

SEQ ID NO: 23 - DNA, 20 bp, synthetic construct
  source 1..20: mol_type = other DNA
cctacgggaggcagcagtag

SEQ ID NO: 24 - DNA, 23 bp, synthetic construct
  source 1..23: mol_type = other DNA
caacagagctttacgatccgaaa

SEQ ID NO: 25 - DNA, 20 bp, synthetic construct
  source 1..20: mol_type = other DNA
ttttgatgggtcttgactgg

SEQ ID NO: 26 - DNA, 22 bp, synthetic construct
  source 1..22: mol_type = other DNA
tttattactgtgccgtgttagc

SEQ ID NO: 27 - DNA, 24 bp, synthetic construct
  source 1..24: mol_type = other DNA
acaattccttgagttccatccaag

SEQ ID NO: 28 - DNA, 18 bp, synthetic construct
  source 1..18: mol_type = other DNA
tggtctgctgcctgttgc

SEQ ID NO: 29 - DNA, 20 bp, synthetic construct
  source 1..20: mol_type = other DNA
tgctctaggtgctgggcaag

SEQ ID NO: 30 - DNA, 20 bp, synthetic construct
  source 1..20: mol_type = other DNA
gagctcctccgattcctcca

SEQ ID NO: 31 - DNA, 25 bp, synthetic construct
  source 1..25: mol_type = other DNA
ttatttcgtgatagtttgcttgcat

SEQ ID NO: 32 - DNA, 21 bp, synthetic construct
  source 1..21: mol_type = other DNA
caagcgctcaaacagctcttg

SEQ ID NO: 33 - DNA, 23 bp, synthetic construct
  source 1..23: mol_type = other DNA
agcaatgcagccaatctacaaat

SEQ ID NO: 34 - DNA, 22 bp, synthetic construct
  source 1..22: mol_type = other DNA
acgaactttgccgttattgtca

SEQ ID NO: 35 - DNA, 29 bp, synthetic construct
  source 1..29: mol_type = other DNA
ggtttaacgtcaaaattagctgtattagc

SEQ ID NO: 36 - DNA, 20 bp, synthetic construct
  source 1..20: mol_type = other DNA
ctcaaccaaccgccactgtt

SEQ ID NO: 37 - DNA, 20 bp, synthetic construct
  source 1..20: mol_type = other DNA
atggcggttatggacacgtt

SEQ ID NO: 38 - DNA, 20 bp, synthetic construct
  source 1..20: mol_type = other DNA
tttggccaccttgaacacct

SEQ ID NO: 39 - DNA, 20 bp, synthetic construct
  source 1..20: mol_type = other DNA
atattttagggcgcggagca

SEQ ID NO: 40 - DNA, 20 bp, synthetic construct
  source 1..20: mol_type = other DNA
gcttgttccgtgatttgggt

SEQ ID NO: 41 - DNA, 20 bp, synthetic construct
  source 1..20: mol_type = other DNA
ccgcctatcaaccagaagca

SEQ ID NO: 42 - DNA, 20 bp, synthetic construct
  source 1..20: mol_type = other DNA
gctcccttttgaccaccact

(19) United States
(12) Patent Application Publication
(10) Pub. No.: US 2025/0195613 A1
(43) Pub. Date: Jun. 19, 2025

(54) COMPOSITIONS AND METHODS FOR REDUCING BIOFILM FORMATION

(71) Applicants: Trustees of Tufts College, MEDFORD, MA (US); MASSACHUSETTS INSTITUTE OF TECHNOLOGY, Cambridge, MA (US)

(72) Inventors: David L. Kaplan, Concord, MA (US); Katharina Ribbeck, Cambridge, MA (US); Laura L. Kiessling, Cambridge, MA (US); Jugal Kishore Sahoo, Belmont, MA (US); Caroline Andrea Werlang, Cambridge, MA (US)

(21) Appl. No.: 18/970,231

(22) Filed: Dec. 5, 2024

Related U.S. Application Data
(63) Continuation of application No. PCT/US23/68204, filed on Jun. 9, 2023.
(60) Provisional application No. 63/350,859, filed on Jun. 9, 2022.

(51) Int. Cl. A61K 38/17; A61K 9/00; A61K 35/744; C07K 14/435

(52) U.S. Cl. CPC A61K 38/1767; A61K 9/0053; A61K 35/744; C07K 14/43586

(57) ABSTRACT

Compositions and methods for reducing or eliminating biofilm formation are disclosed. The compositions include an O-glycan substituted silk fibroin with a predetermined degree and distribution of O-glycan substitution. The O-glycan substituted silk fibroin is not cytotoxic to the population of biofilm-forming microbes, but it reduces or prevents biofilm formation, nonetheless.

### CROSS-REFERENCE TO RELATED APPLICATIONS

This application is a continuation of PCT International Application Serial Number PCT/US2023/068204 (Attorney Docket No. 2095.0547), filed on Jun. 9, 2023, and published as WO 2023/240237. PCT International Application Serial Number PCT/US2023/068204 is related to, and claims priority to and the benefit of, U.S. Provisional Application No. 63/350,859 (Attorney Docket No. 2095.0411), filed on Jun. 9, 2022, entitled “COMPOSITIONS AND METHODS FOR REDUCING BIOFILM FORMATION”. Each of the aforementioned patent applications are incorporated herein by reference in their entireties for all purposes.

### STATEMENT REGARDING FEDERALLY FUNDED RESEARCH

This invention was made with government support under grants EB017755 and EB027062 awarded by the National Institutes of Health. The government has certain rights in the invention.

### BACKGROUND

In some environments, reducing or eliminating biofilm formation can be easily achieved simply by ensuring that no microbes can grow at all in an environment. The more challenging situation is reducing or eliminating biofilm formation in environments where microbes are intended to grow, or even environments that are tailored to be as close to ideal as possible for growth (e.g., bioreactors). Most challenging is the circumstance where a biofilm-causing microbe is essential to the process at interest. In these circumstances, it may be imperative that the biofilm-causing microbe maintain viability and/or activity.

A need exists for additional compositions and methods for reducing biofilm formation. A need exists for compositions and methods that can achieve this without negatively impacting specific bacterial populations and/or the overall bacterial population.

### SUMMARY

With the global proliferation of antimicrobial resistance, new strategies for preventing and treating harmful infections are needed.

In one aspect, the present disclosure provides a liquid composition having adequate biofilm-forming microbial population to support formation of a biofilm but having suppressed formation of the biofilm. The liquid composition includes water, a population of biofilm-forming microbes including optionally bacteria or yeast, and an O-glycan substituted silk fibroin. The O-glycan substituted silk fibroin has a predetermined degree and distribution of O-glycan substitution. The O-glycan substituted silk fibroin is not cytotoxic to the population of biofilm-forming microbes. A comparison liquid composition that lacks the O-glycan substituted silk fibroin but is otherwise identical to the liquid composition exhibits a comparison degree of formation of the biofilm under biofilm-promoting conditions. The liquid composition exhibits a degree of formation of the biofilm under the biofilm-promoting conditions. The degree of formation of the biofilm is less than the comparison degree of formation of the biofilm.

In another aspect, the present disclosure provides a biofilm-resistant coating comprising, consisting essentially of, or consisting of the O-glycan substituted silk fibroin.

In a further aspect, the present disclosure provides a method of making an O-glycan substituted silk fibroin. The method includes: selecting reaction conditions that are intended to provide the O-glycan-substituted silk fibroin with a predetermined degree and distribution of O-glycan substitution, wherein the predetermined degree and distribution of O-glycan substitution exhibits a degree of formation of a biofilm under biofilm-promoting conditions; and covalently modifying silk fibroin to contain a plurality of O-glycan substitutions using the reaction conditions, thereby providing the O-glycan-substituted silk fibroin.

In yet another aspect, the present disclosure provides a method of making a liquid composition. The method includes combining water, a population of microbes, an O-glycan-substituted silk fibroin having a degree of substitution to form the liquid composition.

In yet a further aspect, the present disclosure provides a method of using an O-glycan-substituted silk fibroin. The method includes introducing an O-glycan substituted silk fibroin having a predetermined degree of O-glycan substitution into a liquid composition comprising a population of bacteria including optionally bacteria or yeast.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE DRAWINGS

The disclosure and the following detailed description of certain embodiments thereof may be understood by reference to the following figures:

FIGS. 1A-C is a set of diagrams showing that engineered glycosilk SF-(S)-GalNAc reduces biofilm formation in Streptococcus mutans without killing.

FIGS. 2A-D is a set of diagrams showing that biofilm reducing capacities of glycosilk is specific to GalNAc grafting.

FIGS. 3A-B are a pair of diagrams showing that biofilm reduction depends on concentration and grafting density of GalNAc on silk fibroin biopolymer.

FIG. 4 is a graph showing quantification of the primary amine content of different protein polymers by a 2,4,6-Trinitrobenzene Sulfonic Acid (TNBSA) assay.

FIG. 5 is a graph showing the zeta potential (mV) of different protein polymers at 1 wt % in a 4-(2-hydroxyethyl)-1-piperazineethanesulfonic acid (HEPES) buffer (pH 7.4).

FIG. 6A is a plot of Fourier transform Infra-red (FT-IR) spectra of silk-sugar glycopolymers.

FIG. 6B is a plot of beta-sheet quantification for sugar-silk glycopolymers.

FIG. 7 is a graph showing that SF-(S)-NeuNAc is toxic to S. mutans in a concentration dependent manner.

### DETAILED DESCRIPTION

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; (v) where ranges are provided, endpoints are included; (vi) when used herein, the term “comprising” also expressly contemplates the use of the terms “consisting essentially of” and “consisting” in its place, unless the context clearly dictates otherwise, using the definitions consistent with United States patent law.

Biocompatible: the term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, including but not limited to, less than or equal to 15%, 10%, 5%, or 1% cell death.

Biodegradable: as used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively, or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid) (PLA), poly(glycolic acid) (PGA), poly(lactic-co-glycolic acid) (PLGA), and copolymers with PEG, polyanhydrides, poly(ortho) esters, polyesters, polyurethanes, poly(butyric acid), poly(valeric acid), poly(caprolactone), poly(hydroxyalkanoates), poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Compaction: as used herein, the term “compaction” refers to a process by which a material progressively loses its porosity due to the effects of loading.

Composition: as used herein, may be used to refer to a discrete physical entity that comprises one or more specified components. In general, unless otherwise specified, a composition may be of any form—e.g., gas, gel, liquid, solid, etc. In some embodiments, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Cytotoxic: as used herein, has the opposite meaning of biocompatible. A skilled artisan will recognize that there are a variety of means of assessing cytotoxicity with respect to a specific bacteria or population of bacteria, including some of the methods described herein.

Fibroin: As used herein, the term “fibroin” includes silkworm silk fibroin and insect or spider silk protein (Lucas et al, Adv. Protein Chem 13:107-242 (1958)). Any type of silk fibroin can be used according to aspects of the present invention. There are many different types of silk produced by a wide variety of species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. In some embodiments, fibroin is obtained from a solution containing a dissolved silkworm silk or spider silk. The silkworm silk protein is obtained, for example, from Bombyx mori, and the spider silk is obtained from Nephila clavipes. Other silks include transgenic silks, genetically engineered silks (recombinant silk), such as silks from bacteria, yeast, mammalian cells, transgenic animals, or transgenic plants, and variants thereof. See for example, WO 97/08315 and U.S. Pat. No. 5,245,012, content of both of which is incorporated herein by reference in its entirety. In some embodiments, silk fibroin can be derived from other sources such as spiders, other silkworms, bees, synthesized silk-like peptides, and bioengineered variants thereof. In some embodiments, silk fibroin can be extracted from a gland of silkworm or transgenic silkworms. See for example, WO2007/098951, content of which is incorporated herein by reference in its entirety. Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Ala-rich” and “Gly-rich” hydrophobic blocks are typically separated by segments of amino acids with bulky side-groups (e.g., hydrophilic spacers). In some embodiments, core repeat sequences of the hydrophobic blocks of fibroin can be represented by the following amino acid sequences and/or formulae: (GAGAGS)5-15 (SEQ ID NO: 1); (GX)5-15 (X=V, I, A) (SEQ ID NO: 2); GAAS (SEQ ID NO: 3); (S1-2A11-13) (SEQ ID NO: 4); GX1-4 GGX (SEQ ID NO: 5); GGGX (X=A, S, Y, R, D V, W, R, D) (SEQ ID NO: 6); (Sl-2Al-4)l-2 (SEQ ID NO: 7); GLGGLG (SEQ ID NO: 8); GXGGXG (X=L, I, V, P) (SEQ ID NO: 9); GPX (X=L, Y, I); (GP(GGX)l-4 Y)n (X=Y, V, S, A) (SEQ ID NO: 10); GRGGAn (SEQ ID NO: 11); GGXn (X=A, T, V, S); GAG (A)6-7GGA (SEQ ID NO: 12); and GGX GX GXX (X=Q, Y, L, A, S, R) (SEQ ID NO: 13). In some embodiments, a fibroin peptide can contain multiple hydrophobic blocks, e.g., 3, 4, 5, 6, 7, 8, 9, 10, 11, 12, 13, 14, 15, 16, 17, 18, 19 and 20 hydrophobic blocks within the peptide. In some embodiments, a fibroin peptide can contain between 4-17 hydrophobic blocks. In some embodiments of the invention, a fibroin peptide comprises at least one hydrophilic spacer sequence (“hydrophilic block”) that is about 4-50 amino acids in length. Non-limiting examples of the hydrophilic spacer sequences include: TGSSGFGPYVNGGYSG (SEQ ID NO: 14); YEYAWSSE (SEQ ID NO: 15); SDFGTGS (SEQ ID NO: 16); RRAGYDR (SEQ ID NO: 17); EVIVIDDR (SEQ ID NO: 18); TTIIEDLDITIDGADGPI (SEQ ID NO: 19) and TISEELTI (SEQ ID NO: 20). In certain embodiments, a fibroin peptide can contain a hydrophilic spacer sequence that is a derivative of any one of the representative spacer sequences listed above. Such derivatives are at least 75%, at least 80%, at least 85%, at least 90%, or at least 95% identical to any one of the hydrophilic spacer sequences. In some embodiments, a fibroin peptide suitable for the present invention contains no spacer. Silks are generally fibrous proteins and characterized by modular units linked together to form high molecular weight, highly repetitive proteins. These modular units or domains, each with specific amino acid sequences and chemistries, are thought to provide specific functions. For example, sequence motifs such as poly-alanine (poly A) and poly-alanine-glycine (poly-AG) are inclined to be beta-sheet-forming; GXX motifs contribute to 31-helix formation; GXG motifs provide stiffness; and GPGXX (SEQ ID NO: 22) contributes to beta-spiral formation. These are examples of different components in various silk structures whose positioning and arrangement are tied with the end material properties of silk-based materials (reviewed in Omenetto and Kaplan (2010) Science 329:528-531). Also see: WO 2011/130335 (PCT/US2011/032195), the contents of which are incorporated herein by reference.

Improve, increase, or reduce: as used herein or grammatical equivalents thereof, indicate values that are relative to a baseline measurement, such as a measurement in a similar composition made according to previously known methods.

Physiological conditions: as used herein, has its art-understood meaning referencing conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Reference: as used herein describes a standard or control relative to which a comparison is performed. For example, in some embodiments, a material, article, additive, entity or other sample, sequence or value of interest is compared with a reference or control material, article, additive, entity or other sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Silk is a natural protein fiber produced in a specialized gland of certain organisms. Silk production in organisms is especially common in the Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high-performance fibers.

Silk has been a highly desired and widely used textile since its first appearance in ancient China (see Elisseeff, “The Silk Roads: Highways of Culture and Commerce,” Berghahn Books/UNESCO, New York (2000); see also Vainker, “Chinese Silk: A Cultural History,” Rutgers University Press, Piscataway, New Jersey (2004)). Glossy and smooth, silk is favored by not only fashion designers but also tissue engineers because it is mechanically tough but degrades harmlessly inside the body, offering new opportunities as a highly robust and biocompatible material substrate (see Altman et al., Biomaterials, 24:401 (2003); see also Sashina et al., Russ. J. Appl. Chem., 79:869 (2006)).

Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

As is known in the art, silks are modular in design, with large internal repeats flanked by shorter (−100 amino acid) terminal domains (N and C termini). Naturally-occurring silks have high molecular weight (200 to 350 kDa or higher) with transcripts of 10,000 base pairs and higher and >3000 amino acids (reviewed in Omenetto and Kaplan (2010) Science 329:528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules.

In general, silk fibroin for use in accordance with the present invention may be produced by any such organism, or may be prepared through an artificial process, for example, involving genetic engineering of cells or organisms to produce a silk protein and/or chemical synthesis. In some embodiments of the present invention, silk fibroin is produced by the silkworm, Bombyx mori. Fibroin is a type of structural protein produced by certain spider and insect species that produce silk. Cocoon silk produced by the silkworm, Bombyx mori, is of particular interest because it offers low-cost, bulk-scale production suitable for a number of commercial applications, such as textile.

Silkworm cocoon silk contains two structural proteins, the fibroin heavy chain (˜350 kDa) and the fibroin light chain (˜25 kDa), which are associated with a family of nonstructural proteins termed sericin, which glue the fibroin brings together in forming the cocoon. The heavy and light chains of fibroin are linked by a disulfide bond at the C-terminus of the two subunits (see Takei, F., Kikuchi, Y., Kikuchi, A., Mizuno, S. and Shimura, K. (1987) 105 J. Cell Biol., 175-180; see also Tanaka, K., Mori, K. and Mizuno, S. 114 J. Biochem. (Tokyo), 1-4 (1993); Tanaka, K., Kajiyama, N., Ishikura, K., Waga, S., Kikuchi, A., Ohtomo, K., Takagi, T. and Mizuno, S., 1432 Biochim. Biophys. Acta., 92-103 (1999); Y Kikuchi, K Mori, S Suzuki, K Yamaguchi and S Mizuno, “Structure of the Bombyx mori fibroin light-chain-encoding gene: upstream sequence elements common to the light and heavy chain,” 110 Gene, 151-158 (1992)). The sericins are a high molecular weight, soluble glycoprotein constituent of silk which gives the stickiness to the material. These glycoproteins are hydrophilic and can be easily removed from cocoons by boiling in water.

As used herein, the term “silk fibroin” refers to silk fibroin protein, whether produced by silkworm, spider, or other insect, or otherwise generated (Lucas et al., 13 Adv. Protein Chem., 107-242 (1958)). In some embodiments, silk fibroin is obtained from a solution containing a dissolved silkworm silk or spider silk. For example, in some embodiments, silkworm silk fibroins are obtained from the cocoon of Bombyx mori. In some embodiments, spider silk fibroins are obtained, for example, from Nephila clavipes. In some embodiments, silk fibroins suitable for use in the invention are obtained from a solution containing a genetically engineered silk harvested from bacteria, yeast, mammalian cells, transgenic animals or transgenic plants. See, e.g., WO 97/08315 and U.S. Pat. No. 5,245,012, each of which is incorporated herein as reference in its entirety.

In some embodiments, a silk solution is used to fabricate compositions of the present invention contain fibroin proteins, essentially free of sericins. In some embodiments, silk solutions used to fabricate various compositions of the present invention contain the heavy chain of fibroin but are essentially free of other proteins. In other embodiments, silk solutions used to fabricate various compositions of the present invention contain both the heavy and light chains of fibroin but are essentially free of other proteins. In certain embodiments, silk solutions used to fabricate various compositions of the present invention comprise both a heavy and a light chain of silk fibroin; in some such embodiments, the heavy chain and the light chain of silk fibroin are linked via at least one disulfide bond. In some embodiments where the heavy and light chains of fibroin are present, they are linked via one, two, three or more disulfide bonds. Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Alanine-rich” hydrophobic blocks are typically separated by segments of amino acids with bulky side-groups (e.g., hydrophilic spacers).

Silk fibroin materials explicitly exemplified herein were typically prepared from material spun by silkworm, Bombyx mori. Typically, cocoons are boiled in an aqueous solution of 0.02 M Na2CO3, then rinsed thoroughly with water to extract the glue-like sericin proteins (this is also referred to as “degumming” silk). Extracted silk is then dissolved in a solvent, for example, LiBr (such as 9.3 M) solution at room temperature. A resulting silk fibroin solution can then be further processed for a variety of applications as described elsewhere herein.

In some embodiments, polymers of silk fibroin fragments can be derived by degumming silk cocoons at or close to (e.g., within 5% around) an atmospheric boiling temperature for at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

As used herein, the phrase “silk fibroin fragments” refers to peptide chains or polypeptides having an amino acid sequence corresponding to fragments derived from silk fibroin protein or variants thereof. In the context of the present disclosure, silk fibroin fragments generally refer to silk fibroin peptide chains or polypeptides that are smaller than the naturally occurring full length silk fibroin counterpart, such that one or more of the silk fibroin fragments within a population or composition are less than 300 kDa. The provided silk fibroin fragments may be degummed under a specific condition (e.g., degumming time and atmospheric boiling temperature or a temperature ranging from 90° C. to 110° C.) to produce silk fibroin fragments having a desired molecular weight. In some embodiments, a silk solution may be produced having silk fibroin with a molecular weight that ranges from 3.5 kDa to 300 kDa, from 50 kDa to 120 kDa, or from 120 kDa to 300 kDa. In some embodiments, the molecular weight is at least 3.5 kDa, or at least 5 kDa, or at least 10 kDa, or at least 20 kDa, or at least 30 kDa, or at least 40 kDa, or at least 50 kDa, or at least 60 kDa, or at least 70 kDa, or at least 80 kDa, or at least 90 kDa, to less than 100 kDa, or less than 110 kDa, or less than 120 kDa, or less than 130 kDa, or less than 140 kDa, or less than 150 kDa, or less than 200 kDa, or less than 250 kDa, or less than 300 kDa. In some cases, the silk fibroin can be a low molecular weight silk fibroin, such as is described in WO 2014/145002, which is incorporated herein in its entirety by reference.

### Compositions and Methods

The present disclosure was motivated by challenges associated with biofilm formation, particularly with formation of biofilms from Streptococcus mutans in subjects that are unable to produce an adequate amount of healthy saliva. In healthy saliva, a gel-forming glycoprotein named mucin reduced biofilm formation from S. mutans. A need exists for synthetic and improved compositions and methods for reducing biofilm formation, particularly from S. mutans. Importantly, in some circumstances, reduction or prevention of biofilm formation can be achieved by killing all bacteria, but that is not possible in many instances where useful bacteria must be kept alive, such as in the gastrointestinal tracts. As such, an additional goal of the compositions and methods developed by the present inventors is the reduction of biofilm formation without significantly impacting overall bacterial population. In other words, the inventive compositions and methods described herein can achieve prevention or reduction of biofilm formation without necessitating the killing of the bacteria from which the biofilm is formed. This has important implications for oral and gastrointestinal health as well as other applications where killing the bacteria is not a satisfactory solution to the problem of biofilm formation.

In response to these needs and in the process of continuing their respective research that has earned them tenured positions at world-leading research institutions (i.e., positions requiring significantly more skill than a person having ordinary skill in the art), the present inventors developed the compositions and methods disclosed herein.

A liquid composition is disclosed. The liquid composition includes water, a population of biofilm-forming bacteria, and an O-glycan-substituted silk fibroin.

The population of biofilm-forming bacteria can comprise or can be entirely composed of Streptococcus mutans. The population of biofilm-forming bacteria can comprise or can be entirely composed of Staphylococcus aureus. The population of biofilm-forming bacteria can comprise or can be entirely composed of Streptococcus sanguinis. The population of biofilm-forming bacteria can comprise or can be entirely composed of other microbes that a skilled artisan would recognize as being suitable for use with the compositions described herein. The populations can range from one bacterium to 10 billion bacteria per milliliter or from 1000 to one billion bacteria per milliliter.

The O-glycan-substituted silk fibroin can be present in the liquid composition in an amount by weight of between 0.5% and 30%, including but not limited to, at least 0.5%, at least 1.0%, at least 1.5%, at least 2.05%, at least 3.0%, at least 4.0%, or at least 4.5%, or at most 30%, at most 25%, at most 20%, at most 15%, or at most 10%.

The O-glycan substitution can be preferentially targeted toward a specific amino acid residue, such as the serine or threonine residue. In other cases, the O-glycan substitution can be preferentially targeted at a different amino acid residue of different residues, depending on the desired degree of coverage and location. In some cases, the O-glycan-substituted silk fibroin comprises O-glycan substitutions on at least 0.5%, at least 1.0%, at least 1.5%, at least 2.5%, at least 4.0%, at least 5.0%, at least 7.0%, at least 8.5%, at least 10.0%, at least 12.0%, at least 17.5%, at least 20.0%, at least 25.0% of amino acid residues in the O-glycan-substituted silk fibroin. In some cases, the O-glycan-substituted silk fibroin comprises O-glycan substitutions on at most 40.0%, at most 35.0%, at most 30.0%, or at most 25.0% of amino acid residues in the O-glycan-substituted silk fibroin. In some cases, the distribution of O-glycan substitution can be tailored by selecting amino acid residues for substitution, however a skilled artisan will recognize that the underlying protein sequence will strongly influence the ability to tailor the distribution (i.e., the locations of the substitutions).

The O-glycan substituents can be bound via a carboxyalkyl linker, such as a carboxybutyl linker. Linkers of this sort may or may not be used with the present disclosure and a skilled artisan will recognize the impact that can result to O-glycan dynamic mobility, packing density, and related biophysical features.

The O-glycan substitution can in some cases be O-linked N-acetyl galactosamine (GalNAc). When linked to the silk fibroin backbone via a serine residue, this produces SF-(S)-GalNAc, as shown below in Example 1. While other O-glycans are contemplated, the results produced by SF-(S)-GalNAc are qualitatively different than other molecules produced and its biofilm reducing capabilities are significantly better than would have been expected. As a result, even if the general concept of O-glycan-substituted silk fibroin is somehow shown to have been contemplated or created before in a very general sense, then the unexpectedly superior performance of O-linked GalNAc was unexpected.

The liquid composition exhibits a degree of formation of a biofilm under biofilm-promoting conditions. A comparison liquid composition that lacks the O-glycan-substituted silk fibroin but is otherwise identical to the liquid composition exhibits a comparison degree of formation of the biofilm under the biofilm-promoting conditions. The degree of formation is less than the comparison degree of formation.

The degree of formation is also favorable when compared with a variety of different performance standards. Some of these properties may be exclusive to compositions where the O-glycan substitution is a GalNAc substitution. A skilled artisan will recognize when these properties are applicable to the broader category of O-glycan-substituted silk fibroin and when they are applicable to the more specific category of GalNAc-substituted silk fibroin.

A mucin-comparison liquid composition lacks the O-glycan-substituted silk fibroin, includes 0.5% by weight of mucin, and is otherwise identical to the liquid composition. In some cases, the mucin is MUC5B. The mucin-comparison liquid composition exhibits a mucin-comparison degree of formation of the biofilm under the biofilm-promoting conditions. The degree of formation is less than the mucin-comparison degree of formation.

A glucosamine-comparison liquid composition lacks the O-glycan-substituted silk fibroin, contains a N-acetyl glucosamine-substituted silk fibroin, and is otherwise identical to the liquid composition. The glucosamine-comparison liquid composition exhibits a glucosamine-comparison degree of formation of the biofilm under the biofilm-promoting conditions. The degree of formation is less than the glucosamine-comparison degree of formation.

A galactose-comparison liquid composition that lacks the O-glycan-substituted silk fibroin, contains galactose-substituted silk fibroin, and is otherwise identical to the liquid composition. The galactose-comparison liquid composition exhibits a galactose-comparison degree of formation of the biofilm under the biofilm-promoting conditions. The degree of formation is less than the galactose-comparison degree of formation.

A glucose-comparison liquid composition that lacks the O-glycan-substituted silk fibroin, contains glucose-substituted silk fibroin, and is otherwise identical to the liquid composition. The glucose-comparison liquid composition exhibits a glucose-comparison degree of formation of the biofilm under the biofilm-promoting conditions. The degree of formation is less than the glucose-comparison degree of formation.

The O-glycan-substituted silk fibroin has a predetermined degree and distribution of O-glycan substitution. In some cases, the predetermined degree and distribution is selected by choosing amino acids for modification and selecting amino acids that provide the desired coverage and locations along the protein backbone.

The O-glycan-substituted silk fibroin is not cytotoxic to the population of biofilm-forming bacteria. In some cases, the O-glycan-substituted silk fibroin is not cytotoxic to other cell types, including mammalian cells.

A biofilm-resistant coating is also disclosed. The coating can comprise, consist essentially of, or consist of the O-glycan-substituted silk fibroin disclosed herein. In some cases, the coating can include other components, as would be appreciated by a person having ordinary skill in the anti-biofilm coating arts.

In some cases, the only plasticizer present in the compositions disclosed herein is water. Other plasticizers, such as glycerol or others, could be present.

The biofilm-resistant coating can have improved performance at resisting the formation and growth of biofilms, in a similar fashion as described above with respect to the liquid composition and blow with respect to the artificial saliva. However, in this case, the comparison is between performance of the inventive coating versus performance of a coating that does not possess the inventive anti-biofilm properties. As one example possibility, contacting the inventive biofilm-resistant coating with a bacterial liquid composition comprising water and the population of biofilm-forming bacteria will cause a degree of formation of a biofilm under biofilm-promoting conditions. In a similar fashion, contacting a polystyrene surface that is otherwise the same as the surface of the biofilm-resistant coating (in many cases, the surface underlying the coating is polystyrene, so in those cases, the comparison polystyrene surface is the same surface but without the coating) with the bacterial liquid composition will cause a polystyrene-comparison degree of formation of the biofilm. The degree of formation of the biofilm is less than the polystyrene-comparison degree of formation of the biofilm. That is, the anti-biofilm coating is resistant to the formation of biofilms when applied to surfaces that otherwise would allow the formation of biofilms.

An artificial saliva is disclosed. The artificial saliva can be the same as the liquid composition described herein, except that in some cases it may not possess the population of bacteria therein (i.e., the bacteria are in the subject's oral cavity, so they will become part of the composition upon administration of the artificial saliva to the oral cavity).

The liquid composition and/or the artificial saliva can have a viscosity that roughly mirrors that of native saliva, both at a high and low shear rate. In some cases, the viscosity can be at least 2 mPa, at least 3 mPa, or at least 4 mPa at a 90 s⁻¹ shear rate. In some cases, the viscosity can be at most 6 mPa, at most 5 mPa, or at most 4 mPa at a 90 s⁻¹ shear rate. In some cases, the viscosity can be at least 50 mPa, at least 60 mPa, at least 70 mPa, or at least 80 mPa at a 1 s⁻¹ shear rate. In some cases, the viscosity can be at most 100 mPa, at most 90 mPa, at most 80 mPa, or at most 75 mPa at a 1 s⁻¹ shear rate. A skilled artisan will recognize that other viscosities at other shear rates may be representative of roughly the same viscosity as native saliva and will appreciate that those viscosities may be usable here.

This liquid composition can generally be free of organic solvents, such as methanol or ethanol. However, there can be circumstances when organic solvents may be present or even necessary. In some cases, organic solvents may be introduced in order to induce a desired crystallinity in the O-glycan-substituted silk fibroin. As evidenced by the experimental results in Example 1, and without wishing to be bound by any particular theory, it is believed that the O-glycan substitution does not significantly alter the crystallinity of the silk fibroin, so it is expected that the O-glycan-substituted silk fibroin can be induced to adopt a desired degree of crystallinity that is roughly the same as any desired degree of crystallinity that is achievable by native silk fibroin or unsubstituted silk fibroin.

Biofilm-forming conditions, as used herein, refer to a set of conditions under which a skilled artisan would reasonably expect a biofilm to form. Biofilm-forming conditions can include, but are not limited to, conditions in which bacteria grow in liquid, in gels, or on surfaces when significant moisture is present. Surfaces can include parts of the human body, like skin, bones, teeth, or gums, as well as man-made materials, like plastics, including polystyrene. Biofilm-forming environments can range in temperature from 5 to 45 degrees Celsius (preferred range 30-40 degrees C.). These can be high nutrient environments like the mouth, or gut, but biofilms can also grow in low-nutrient environments.

The biofilm content from a given liquid composition is measured using techniques understood to those having ordinary skill in the art, including those disclosed herein. One specific example is described in Example 1. Changes in biofilm formation are generally measured as a percent change when compared to some reference measurement. In this way, the inventive compositions described herein have a variety of improved properties relative to a variety of reference measurements. The list of improved properties described herein is not intended to be exhaustive and the compositions and methods disclosed may also include other properties that are not explicitly disclosed or mentioned as being specifically observed at the present time.

The present disclosure provides a method of making an O-glycan-substituted silk fibroin. The method includes: selecting reaction conditions that are intended to provide an O-glycan-substituted silk fibroin with a predetermined degree and distribution of O-glycan substitutions; and covalently modifying silk fibroin to contain a plurality of O-glycan substitutions using the reaction conditions, thereby providing the O-glycan-substituted silk fibroin.

The present disclosure also provides a method of making a liquid composition. The method includes: combining water, a population of bacteria, and an O-glycan-substituted silk fibroin having a degree of O-glycan substitution to form the liquid composition.

The present disclosure provides a method of using an O-glycan-modified silk fibroin. The method includes introducing an O-glycan-substituted silk fibroin having a predetermined degree of O-glycan substitution into a liquid composition comprising a population of bacteria.

The present disclosure further provides a method of treating a subject to reduce oral biofilm formation. The method includes: administering to the subject oral cavity the O-glycan-substituted silk fibroin and/or the artificial saliva disclosed herein. In some cases, the performance of such a method can be estimated by using an in vitro mimic for the oral cavity.

Without wishing to be bound by any particular theory, the screening for biofilm reduction illustrated that certain reductions were caused by population redistribution, namely reduction in biofilm that does not result from bacterial death, but rather from the bacteria no longer forming biofilms.

### Example 1

Example 1 includes the experimental results as described below.

### Materials and Methods

### Strains and Reagents

The bacterial strain S. mutans UA159 (ATCC, 700610) was originally isolated from a child with active cavities. Bacteria were cultivated in Bacto Todd-Hewitt broth (TH; BD, 249240) or brain-heart infusion (BHI; BD, 237500) 1.5% agar plates at 37° C. with 5% CO₂. To promote biofilm formation, S. mutans was grown in 25% TH supplemented with 1% sucrose.

Mucin Purification from Pooled Saliva

Submandibular saliva was collected from informed consenting volunteers as described previously. See Frenkel, E. S. & Ribbeck, K., “Salivary mucins in host defense and disease prevention,” J. Oral Microbiol. 7, (2015), which is incorporated herein in its entirety by reference for all purposes. Saliva donations from five donors were pooled before MUC5B was purified using liquid chromatography as described previously. See Werlang, C. A. et al., “Mucin O-glycans suppress quorum-sensing pathways and genetic transformation in Streptococcus mutans,” Nat. Microbiol. 1-10 (2021) doi: 10.1038/s41564-021-00876-1, which is incorporated herein in its entirety by reference for all purposes. Purified MUC5B was flash cooled, lyophilized, and stored at −80° C. Before use, MUC5B was rehydrated in Milli-Q-purified water and agitated at 4° C. overnight. Protocols involving samples from human participants were approved by the Massachusetts Institute of Technology's Committee on the Use of Humans as Experimental Subjects.

MUC5B contains Thr (T), 11% Ser(S), 10% Pro (P), 9% Ala (A), 5% Cys (C), and 44% others.

### Biofilm Growth and Biomass Assays

Overnight cultures of S. mutans UA159 were inoculated from glycerol stocks and grown for 12-18 h in 50% TH at 37° C. with 5% CO₂. Biofilms were prepared by diluting overnight cultures 1:20 into 50-100 μl of media (TH25% with 1% sucrose) and any polymer solution in a 96-well polystyrene plate for a starting inoculum of 10⁷-10⁸ CFU per ml. The cultures were then incubated statically for 5 hours at 37° C. with 5% CO₂.

To measure total growth and relative biofilm formation, the number of cells in the biofilm and in suspension were counted using a colony forming unit (CFU) assay as previously described. See Werlang, C. A. et al., “Mucin O-glycans suppress quorum-sensing pathways and genetic transformation in Streptococcus mutans,” Nat. Microbiol. 1-10 (2021) doi: 10.1038/s41564-021-00876-1, which is incorporated herein in its entirety by reference for all purposes. Briefly, the supernatant was removed and transferred to a new 96-well plate, the biofilm was washed twice with 100 μl of phosphate-buffered saline (PBS), and the washes were added to the supernatant. The biofilm was then detached and resuspended in 100 μl of PBS by vigorously scraping with a pipette tip for 30 s. Each culture fraction was then serially diluted and plated on BHI agar. Colonies were counted after 24 h growth at 37° C. with 5% CO₂. The fraction biofilm was calculated as (biofilm CFU per ml)/(biofilm CFU per ml+supernatant CFU per ml). The total growth was calculated as biofilm CFU per ml+supernatant CFU per ml. All of the conditions had at least three biological replicates (individual data points shown on graphs).

Total biofilm biomass was measured using crystal violet staining. The supernatant was aspirated, and the biofilm was washed twice with 100 μl of PBS. Then 100 μl of 0.1% crystal violet was added to each well and incubated for 15 minutes at room temperature. The crystal violet solution was removed, and the stained biofilms were washed five times with 200 μl of distilled water. Finally, 100 μl of 30% acetic acid was added to each well to extract the absorbed crystal violet, and the OD was measured at 580 nm on a microtiter plate reader.

### Polymer Attachment Assay

Bacterial attachment to polymer coating was assayed as previously described. See Co, J. Y., Crouzier, T. & Ribbeck, K., “Probing the Role of Mucin-Bound Glycans in Bacterial Repulsion by Mucin Coatings,” Adv. Mater. Interfaces 2, 1500179 (2015), which is incorporated herein in its entirety by reference for all purposes. Briefly, coatings were prepared by incubating 100 μl of polymer solutions (2 mg per ml in Milli-Q purified water) on 96-well polystyrene plates for 2 h at 37° C., followed by three washes with 200 μl of sterile-filtered distilled water. Overnight cultures of S. mutans were centrifuged and resuspended in sterile PBS at an OD₆₀₀ of 0.4, and 50 μl of the washed culture were added to each well. The bacteria were incubated on the coating for 1 hour at 37° C. To measure cell attachment, unattached bacteria were aspirated, wells were washed three times with 200 μl of PBS, and the remaining bacteria were detached with a pipette and resuspended in PBS before being quantified by a CFU assay.

### Confocal Microscopy

For confocal microscopy, S. mutans biofilms were prepared as described above on 96-well glass-bottom plates with 5 nM of SYTO9 green fluorescent nucleic acid stain (ThermoFisher, S34854) added to the cultures. Cultures were imaged on a confocal laser-scanning microscope (LSM 800; Zeiss) with a 63×/1.4 NA oil immersion objective with a step size of 0.5 μm. The excitation wavelength for SYTO9 was 488 nm. At least three stacks were recorded for each well, and at least three independent wells were analyzed for each condition. Images were analyzed with Zeiss ZEN 2.1 imaging software (Thornwood, NY, USA).

### Gene Expression Analysis

A table of the primers used in this study is provided in Table 1, which shows Primers used for RT-qPCR. Primers that are new to this study were designed using Primer-BLAST. See Ye, J. et al., “Primer-BLAST: a tool to design target-specific primers for polymerase chain reaction,” BMC Bioinformatics 13, 134 (2012), which is incorporated herein in its entirety by reference for all purposes. Gene expression analysis was performed using quantitative PCR with reverse transcription (RT-qPCR) as previously described. See Werlang, C., Cárcarmo-Oyarce, G. & Ribbeck, K., “Engineering mucus to study and influence the microbiome,” Nat. Rev. Mater. 1 (2019) doi: 10.1038/s41578-018-0079-7, which is incorporated herein in its entirety by reference for all purposes. Briefly, biofilms were grown as described above, but in PCR tubes. After 2 h of growth, cells were centrifuged, the supernatant was removed, and the pellet was flash cooled in liquid nitrogen. Total nucleic acids were then extracted using the MasterPure Complete RNA Purification kit (Lucigen). Genomic DNA was removed using the Turbo DNA-free kit (Ambion). Total RNA was measured using an Agilent 2100 Bioanalyzer (Agilent Technologies) and stored at −80° C. cDNA was synthesized with the Protoscript II First-Strand cDNA Synthesis kit (New England Biolabs). About 2 ng of cDNA was used as a template for qPCR using SYBR Green Master Mix (Thermo Fisher Scientific) performed using the Cycler 480 II Real-time PCR Machine (Roche). Gene expression changes were calculated as the mean change in qPCR cycle threshold compared to 16S rRNA (ΔCₜ) and are reported as log 2 [fold change]=ΔC_{t_media}−ΔC_{t_sample}. Each sample was analyzed with at least three technical replicates.

TABLE 1

| Gene | FWD | REV | Source |
|---|---|---|---|
| 16S | CCTACGGGAGGCAG | CAACAGAGCTTTAC | Werlang et. |
| rRNA | CAGTAG | GATCCGAAA | al. 2021 |
| comS | TTTTGATGGGTCTT | TTTATTACTGTGCC | Wenderska |
| | GACTGG | GTGTTAGC | 2012 |
| comDE | ACAATTCCTTGAGT | TGGTCTGCTGCCTG | Liu et. al. |
| (blpHR) | TCCATCCAAG | TTGC | 2011 |
| cipB | TGCTCTAGGTGCTG | GAGCTCCTCCGATT | Khan et. al. |
| (nlmC) | GGCAAG | CCTCCA | 2016 |
| comX | TTATTTCGTGATAG | CAAGCGCTCAAACA | Okinaga et. |
| (sigX) | TTTGCTTGCAT | GCTCTTG | al. 2010 |
| gtfB | AGCAATGCAGCCAA | ACGAACTTTGCCGT | Shemesh |
| | TCTACAAAT | TATTGTCA | et. al. 2007 |
| gtfC | GGTTTAACGTCAAA | CTCAACCAACCGCC | Gabe et. al. |
| | ATTAGCTGTATTAG | ACTGTT | 2019 |
| | C | | |
| gbpB | ATGGCGGTTATGGA | TTTGGCCACCTTGA | Gabe et. al. |
| | CACGTT | ACACCT | 2019 |
| gbpC | ATATTTTAGGGCGC | GCTTGTTCCGTGAT | This study |
| | GGAGCA | TTGGGT | |
| gbpD | CCGCCTATCAACCA | GCTCCCTTTTGACC | This study |
| | GAAGCA | ACCACT | |

Each source identified in this table is incorporated herein by reference for all purposes.

### Extraction of Aqueous Silk Solution

Silk fibroin (SF) solutions were extracted using reported protocol. Briefly, 5 grams of B. mori silkworm (Tajima Shoji Co. Ltd., Yokohama, Japan) cut cocoons are extracted in 2 L (liter) of 0.02 M Na₂CO₃ solution (Sigma-Aldrich, St. Louis, MO) in a glass beaker for 60 minutes to remove the sericin protein coating. Degummed silk was collected and rinsed with deionized water (DI) in a 4 L bucket (20 minutes, 3 times), followed by drying at room temperature in a fume hood overnight. The dried degummed silk fibroin fibers were solubilized in 9.3 M Lithium Bromide (LiBr) (Sigma-Aldrich, St. Louis, MO) solution, in a preheated oven at 60° C. for 4 h. After 4 h (hours), light brown color SF solution was obtained which was dialyzed against 4 L of deionized (DI) water in a bucket with six water changes for 48 h (water changes at 1, 2, 4, 24, 36, and 48 hours). The dialysis was performed with a dialysis tube (3,500 MWCO, Thermo Fisher Scientific, Rockford, IL). After 48 h, the dialyzed silk solution was centrifuged (9,000 RPM, 20 min, 4° C., 2 times) to remove insoluble solid aggregates. The concentration of the regenerated SF solution was calculated by drying a known mass of the aqueous silk solution in a weighing boat in an oven at 60° C. overnight and assessing the mass of the remaining solid film. The aqueous SF solution was stored in the refrigerator at 4° C. until further use.

Silk fibroin contains 46% Gly (G), 30% Ala (A), 12% Ser(S), 5% Tyr (Y), 0.6% Glu (E), 0.5% Asp (D), and 6% other.

### Synthesis of SF(S)-COOH

Aqueous silk fibroin solution was carboxylated by nucleophilic substitution in a highly alkaline reaction environment, in presence of chloroacetic acid (Sigma-Aldrich, St. Louis, MO) at pH ˜13.5. Briefly, the pH of the 1M Chloroacetic acid was adjusted by adding freshly prepared 10M sodium hydroxide (NaOH) solution to raise pH to 13.3-13.5. At pH 13.5, reconstituted silk fibroin solution (0.6 wt %) was added to the mixture. Addition of SF solutions might decrease the pH, which was further adjusted to pH 13.5, by dropwise addition of 10 M NaOH solution. The solution was allowed to stir gently for 1 h at RT. After 1 h, sodium phosphate monobasic (NaH₂PO₄) (Sigma-Aldrich, St. Louis, MO; Lot #015K0024, 4 mg/ml) was added to the mixture and stirred. Addition of NaH₂PO₄ decreases the pH of the mixture. The pH of the solution was adjusted to 7-7.5 by slow dropwise addition of 10M Hydrochloric acid (HCl) (Sigma-Aldrich, St. Louis, MO) solution. At pH 7-7.5, the reaction mixture was stirred for 30 minutes at RT. After 30 minutes, the carboxylated silk solution was dialyzed against DI water for 72 h with six water changes (1 h, 2 h, 4 h, 24 h, 48 h, and 72 h) to remove byproducts and impurities. The dialysis was performed with a dialysis tube (3,500 MWCO, Thermo Fisher Scientific, Rockford, IL) in a 4 L (liter) bucket with gentle stirring. After dialysis, the carboxy-modified silk solutions were filtered in a sterile cell strainer with 40 μm mesh size (Thermo Fisher Scientific, Rockford, IL). After filtration, the solutions were frozen at −80° C. overnight followed by lyophilizing for at least 72 h. The lyophilized powders were collected and stored at 4° C. in a refrigerator until further use.

### Synthesis of SF(S)-EDA

The carboxylated silk fibroin (SF(S)-COOH) was covalently conjugated with primary amines of ethylene diamine (EDA) hydrochloride (Sigma-Aldrich, St. Louis, MO) by carbodiimide coupling in the presence of N-3-Dimethyl amino propyl-N′-ethyl carbodiimide (EDC) hydrochloride, and N-HydroxySuccinimide (NHS) (Sigma-Aldrich, St. Louis, MO). Briefly, 2 wt % of the SF(S)-COOH solution was dissolved in 0.1M MES (2-(N-morpholino) ethanesulfonic acid) buffer at pH 6. EDA (10×) was weighed and pre-dissolved in Ultrapure distilled water (Thermo-Fisher Scientific, Waltham, MA) and added to the SF(S)-COOH solution. The pH was readjusted to 6 by dropwise addition of freshly prepared 1M sodium hydroxide (NaOH) solution. EDC (10×) and NHS (10×) (pre-dissolved in MES buffer, pH 6) were added to the reaction mixture at pH 6. The final MES buffer concentration of the reaction mixture was adjusted to 0.05M by addition of ultrapure distilled water. The reaction was allowed to stir gently at RT for 18 h. After the reaction got over, the aggregates were filtered in a sterile cell strainer with 40 μm mesh size (Thermo Fisher Scientific, Rockford, IL) and dialyzed against DI water for at least 72 h with six water changes (1, 2, 4, 24, 48, and 72 h). The dialysis was performed with a dialysis tube (3500 MWCO, Thermo Fisher Scientific, Rockford, IL). After dialysis, the solutions were frozen at −80° C. overnight followed by lyophilizing for 72 h. The lyophilized powders were stored at 4° C. until further use.

### Synthesis of SF(S)-GalNAc

The aminated SF solution (SF(S)-EDA) were conjugated with carboxylic acid moieties of 4-carboxybutyl N-acetyl-β-D-galactosaminide (β-GalNAc-Bu-COOH) (Sussex Research, Ottawa, Canada) by carbodiimide coupling in presence of N-(3-dimethylaminopropyl)-N′-ethylcarbodiimide hydrochloride (EDC) and N-hydroxy succinimide (NHS) (Sigma-Aldrich, St. Louis, MO). Briefly, 2 wt % of the SF(S)-EDA was dissolved in 0.1M MES (2-(N-morpholino) ethanesulfonic acid) buffer at pH 6. β-GalNAc-Bu-COOH (3× times molar excess)) was weighed and pre-dissolved in 0.1M MES buffer and pH was readjusted 6 by dropwise addition of freshly prepared 1M NaOH solution. EDC (3×) and NHS (3×) were added to β-GalNAc-Bu-COOH at pH 6 to activate the carboxylic acid. The reaction was readjusted to pH 6 and stirred for 30 minutes at RT. After 30 minutes, SF-(S)-EDA solution was dropwise added to the activated solution. The final MES buffer concentration of the reaction mixture was adjusted to 0.05M by addition of ultrapure water. The pH was readjusted to 6 after addition of SF(S)-EDA solution. The reaction was stirred at RT for 18 h. After 18 h, the aggregates were filtered in a sterile cell strainer with 40 μm mesh size (Thermo Fisher Scientific, Rockford, IL) and dialyzed against DI water for at least 72 h with six water changes (1 h, 2 h, 4 h, 24 h, 48 h, and 72 h). The dialysis was performed with a dialysis tube (3,500 MWCO, Thermo Fisher Scientific, Rockford, IL). After dialysis, the solutions were frozen at −80° C. overnight followed by lyophilizing for 72 h. The lyophilized powders were stored at 4° C. until further use.

### Synthesis of SF(S)-GlcNAc

Similar to synthesis of SF(S)-GalNAc as described above, (SF(S)-EDA) was conjugated with carboxylic acid residues of 4-carboxybutyl N-acetyl-β-D-glucosaminide (β-GlcNAc-Bu-COOH) (Sussex Research, Ottawa, Canada) by carbodiimide coupling in presence of EDC and NHS (Sigma-Aldrich, St. Louis, MO). Similar to conjugation method described above for SF(S)-GalNAc, 2 wt % of the SF(S)-EDA was dissolved in 0.1M MES (2-(N-morpholino) ethanesulfonic acid) buffer at pH 6. β-GlcNAc-Bu-COOH (3× molar excess)) was pre-dissolved in 0.1M MES buffer and pH was readjusted 6 by dropwise addition of freshly prepared 1M NaOH solution. EDC (3×) and NHS (3×) were added to β-GlcNAc-Bu-COOH at pH 6 to activate the carboxylic acids. The reaction was readjusted to pH 6 and stirred for 30 minutes at RT. After 30 minutes, SF-(S)-EDA solution was dropwise added to the activated solution. The final MES buffer concentration of the reaction mixture was adjusted to 0.05M by addition of ultrapure water. The pH was readjusted to 6 after addition of SF(S)-EDA solution. The reaction was stirred at RT for 18 h. After 18 h, the aggregates were filtered in a sterile cell strainer with 40 μm mesh size (Thermo Fisher Scientific, Rockford, IL) followed by dialysis against DI water for at least 72 h with six water changes (1 h, 2 h, 4 h, 24 h, 48 h, and 72 h). The dialysis was performed with a dialysis tube (3,500 MWCO, Thermo Fisher Scientific, Rockford, IL). After dialysis, the solutions were frozen at −80° C. overnight followed by lyophilizing for 72 h. The lyophilized powders were stored at 4° C. until further use.

### Synthesis of SF(S)-NeuNAc

Similar to synthesis of SF(S)-GalNAc and SF(S)-NeuNAc as described above, (SF(S)-EDA) was conjugated with carboxylic acid residues of N-Acetylneuraminic acid hydrate (TCI America, Portland, OR) by carbodiimide coupling in presence of EDC and NHS (Sigma-Aldrich, St. Louis, MO). 2 wt % of the SF(S)-EDA was dissolved in 0.1M MES (2-(N-morpholino) ethanesulfonic acid) buffer at pH 6. N-Acetylneuraminic acid hydrate (2× molar excess)) was pre-dissolved in 0.1M MES buffer and pH was readjusted 6 by dropwise addition of freshly prepared 1M NaOH solution. EDC (3×) and NHS (3×) were added to N-Acetylneuraminic acid hydrate at pH 6 to activate the carboxylic acids. The reaction was readjusted to pH 6 and stirred for 30 minutes at RT. After 30 minutes, SF-(S)-EDA solution was dropwise added to the activated solution. The final MES buffer concentration of the reaction mixture was adjusted to 0.05M by addition of ultrapure distilled water. The pH was readjusted to 6 after addition of SF(S)-EDA solution. The reaction was stirred at RT for 18 h. After 18 h, the aggregates were filtered in a sterile cell strainer with 40 μm mesh size (Thermo Fisher Scientific, Rockford, IL) followed by dialysis against DI water for at least 72 h with six water changes (1 h, 2 h, 4 h, 24 h, 48 h, and 72 h). The dialysis was performed with a dialysis tube (3,500 MWCO, Thermo Fisher Scientific, Rockford, IL). After dialysis, the solutions were frozen at −80° C. overnight followed by lyophilizing for 72 h. The lyophilized powders were stored at 4° C. in the refrigerator until further use.

### Synthesis of SF(S)-GlcN

SF(S)-COOH was conjugated with amines of D(+)-Glucosamine Hydrochloride (Sigma-Aldrich, St. Louis, MO) by carbodiimide coupling in presence of EDC and NHS (Sigma-Aldrich, St. Louis, MO). 2 wt % of the SF(S)-COOH solution was dissolved in 0.1M MES (2-(N-morpholino) ethanesulfonic acid) buffer at pH 6. D(+)-Glucosamine Hydrochloride (3×) was weighed and pre-dissolved in ultrapure distilled water (Thermo-Fisher Scientific, Waltham, MA) and added to the SF(S)-COOH solution. The pH was readjusted to 6 by dropwise addition of freshly prepared 1M sodium hydroxide (NaOH) solution. EDC (3×) and NHS (3×) (pre-dissolved in MES buffer, pH 6) were added to the reaction mixture at pH 6. The final MES buffer concentration of the reaction mixture was adjusted to 0.05M by addition of ultrapure distilled water. The reaction was allowed to stir gently at RT for 18 h. The aggregates were filtered, after 18 h, in a sterile cell strainer (40 μm mesh size) (Thermo Fisher Scientific, Rockford, IL) and dialyzed against DI water for at least 72 h with six water changes (1, 2, 4, 24, 48, and 72 h). The dialysis was performed with a dialysis tube (3500 MWCO, Thermo Fisher Scientific, Rockford, IL). After dialysis, the solutions were frozen at −80° C. overnight followed by lyophilizing for 72 h. The lyophilized powders were stored at 4° C. in a refrigerator until further use.

### Synthesis of SF(S)-GalN

SF(S)-GalN was synthesized similar to synthesis of SF(S)-GlcN. (SF(S)-COOH) was conjugated with amines of D(+)-Galactosamine Hydrochloride (Sigma-Aldrich, St. Louis, MO) by carbodiimide coupling in presence of EDC and NHS (Sigma-Aldrich, St. Louis, MO). 2 wt % of the SF(S)-COOH solution was dissolved in 0.1M MES (2-(N-morpholino) ethanesulfonic acid) buffer at pH 6. D(+)-Galactosamine Hydrochloride (3×) was pre-dissolved in ultrapure distilled water (Thermo-Fisher Scientific, Waltham, MA) and added to the SF(S)-COOH solution. The pH was readjusted to 6 by dropwise addition of freshly prepared 1M sodium hydroxide (NaOH) solution. EDC (3×) and NHS (3×) (pre-dissolved in MES buffer, pH 6) were added to the reaction mixture at pH 6. The final MES buffer concentration of the reaction mixture was adjusted to 0.05M by addition of ultrapure distilled water. The reaction was allowed to stir gently at RT for 18 h. The aggregates were filtered, after 18 h, in a sterile cell strainer (40 μm mesh size) (Thermo Fisher Scientific, Rockford, IL) and dialyzed against DI water for at least 72 h with six water changes (1, 2, 4, 24, 48, and 72 h). The dialysis was performed with a dialysis tube (3500 MWCO, Thermo Fisher Scientific, Rockford, IL). After dialysis, the solutions were frozen at −80° C. overnight followed by lyophilizing for 72 h. The lyophilized powders were stored at 4° C. in a refrigerator until further use.

### Synthesis of SF(D, E)-EDA

The regenerated silk fibroin was covalently conjugated with primary amines of ethylene diamine (EDA) hydrochloride (Sigma-Aldrich, St. Louis, MO) by carbodiimide coupling in the presence of EDC, and NHS (Sigma-Aldrich, St. Louis, MO). Briefly, 2 wt % of the SF solution was dissolved in 0.1M MES (2-(N-morpholino) ethanesulfonic acid) buffer at pH 6. EDA (10×) was weighed and pre-dissolved in Ultrapure distilled water (Thermo-Fisher Scientific, Waltham, MA) and added to the aqueous SF solution. The pH was readjusted to 6 by dropwise addition of freshly prepared 1M sodium hydroxide (NaOH) solution. EDC (10×) and NHS (10×) (pre-dissolved in MES buffer, pH 6) were added to the reaction mixture at pH 6. The final MES buffer concentration of the reaction mixture was adjusted to 0.05M by addition of ultrapure distilled water. The reaction was allowed to stir gently at RT for 18 h. After the reaction got over, the aggregates were filtered in a cell strainer (mesh size 40 μm) (Thermo Fisher Scientific, Rockford, IL) and dialyzed against DI water for at least 72 h with six water changes (1, 2, 4, 24, 48, and 72 h). The dialysis was performed with a dialysis tube (3500 MWCO, Thermo Fisher Scientific, Rockford, IL). After dialysis, the solutions were frozen at −80° C. overnight followed by lyophilizing for 72 h. The lyophilized powders were stored at 4° C. until further use.

### Synthesis of SF(D, E)-GalNAc

The aminated SF solution (SF(D, E)-EDA) were conjugated with carboxylic acid moieties of 4-carboxybutyl N-acetyl-β-D-galactosaminide (β-GalNAc-Bu-COOH) (Sussex Research, Ottawa, Canada) by carbodiimide coupling in presence of EDC and NHS (Sigma-Aldrich, St. Louis, MO). Briefly, 2 wt % of the SF(D, E)-EDA was dissolved in 0.1M MES (2-(N-morpholino) ethanesulfonic acid) buffer at pH 6. β-GalNAc-Bu-COOH (3× times molar excess)) was weighed and pre-dissolved in 0.1M MES buffer and pH was readjusted 6 by dropwise addition of freshly prepared 1M NaOH solution. EDC (3×) and NHS (3×) were added to β-GalNAc-Bu-COOH at pH 6 to activate the carboxylic acid. The reaction was readjusted to pH 6 and stirred for 30 minutes at RT. After 30 minutes, the SF(D, E)-EDA solution was dropwise added to the activated solution. The final MES buffer concentration of the reaction mixture was adjusted to 0.05M by addition of ultrapure water. The pH was readjusted to 6 after addition of SF(D, E)-EDA solution. The reaction was stirred at RT for 18 h. After the reaction got over, the aggregates were filtered in a sterile cell strainer with 40 μm mesh size (Thermo Fisher Scientific, Rockford, IL) and dialyzed against DI water for at least 72 h with six water changes (1 h, 2 h, 4 h, 24 h, 48 h, and 72 h). The dialysis was performed with a dialysis tube (3,500 MWCO, Thermo Fisher Scientific, Rockford, IL). After dialysis, the solutions were frozen at −80° C. overnight followed by lyophilizing for 72 h. The lyophilized powders were stored at 4° C. until further use.

### Cytotoxicity of SF-Sugar Glycopolymers, Control Polymers and Sugars

Sugars used in modification of SF and unmodified, carboxylated, aminated and sugar-conjugated SF were dissolved in Dulbecco's Modified Eagle Medium at a concentration of 1 mg/ml and sterile filtered through 0.22 μm filters. L929 fibroblasts from mouse subcutaneous connective tissues (ATCC CCL 1, NCTC Clone 929, of strain L) were seeded in 48 well plates at a density of 2.5×10⁵ cells/cm² and cultured at 37° C. with 5% CO₂ in DMEM growth media supplemented with 10% FBS and 1% Pen-Strep to reach confluency. 48 h after cell seeding, the growth media was replaced with the test solutions. After 24 h metabolic activity of the cells was quantified using alamarBlue viability assay according to the manufacturer's instructions. Relative metabolic activities were calculated by normalization to the SF only solution.

Cytotoxicity Studies of the Extracts from SF-Sugar Conjugate Films

Cytotoxicity of water-insoluble sample films was investigated according to the standard protocol by International Organization for Standardization 10993: Biological Evaluation of Medical Devices, Part 5: Tests for Cytotoxicity: in vitro Methods, which is incorporated herein in its entirety by reference for all purposes. SF and SF-sugar conjugate thin films (thickness <0.5 mm) were prepared by casting of 4.5 wt % aqueous solutions on circular PDMS molds with a diameter of x cm overnight. The dried films are methanol treated overnight in order to render water insolubility by inducing β-sheets. After methanol treatment, the films were dried at room temperature inside a fume hood. Dried SF, SF-sugar glycopolymers prepared in different routes (SF(S)-GalNAc, SF(S)-GlcNAc, SF(S)—NeuNAc, SF(S)-GalN, SF(S)-GlcN, SF(D, E)-GalNAc) and reaction intermediate polymer (SF(S)-COOH, SF(S)-EDA, SF(D, E)-EDA) films and the ISO 10933-5 recommended ZDEC polyurethane films containing 0.1% zinc diethyldithiocarbamate (Hatano Research Institute, FDSC, Japan) as the positive control samples and the high-density polyethylene films (US Pharmacopeia, Rockville, MD) as the negative control samples were ethylene oxide treated for sterilization. The films were incubated for 24 h at 37° C. in 1 ml of Eagle's Minimum Essential Media (EMEM) supplemented with 10% horse serum (Sigma Aldrich, H1138) and 1% Pen-Strep per 6 cm² of total surface area. L929 murine fibroblasts were seeded on 24- and 96-well plates at a density of 2.6×10⁵ cells/cm². 24 h after cell seeding, culture media in 96-well plates were replaced with 0.1 ml of film extracts and the metabolic activity of the cells was quantified at 48 h using MTT assay according to the manufacturer's instructions. 48 h after cell seeding, culture media in 24-well plates were replaced with 0.4 ml of film extracts and the cell morphology was evaluated qualitatively at 72 h and 96 h under a phase contrast microscope.

### Zeta Potential Studies

To investigate the zeta potential of silk-sugar glycopolymers and the control polymers, 10 mg/ml (1 wt %) solutions were prepared in 1×HEPES buffer (syringe-filtered using a 0.22 μm filter). To determine the surface zeta potential of the polymers, electrophoretic mobilities of polymers (n=3) were measured using a NanoBrook ZetaPALS (Brookhaven Instruments, Holtsville, NY) and the zeta potential values were determined using the Smoluchowski model.

### ¹H-Nuclear Magnetic Resonance Spectroscopy (NMR)

The lyophilized powders of all silk-sugar glycopolymers, reaction intermediates and control polymers were reconstituted in deuterated water (D2O) with concentration of 10-15 mg ml⁻¹. The NMR spectroscopy was performed with a 500 MHz Bruker NMR spectrometer. The spectra analysis was performed in Top Spin 3.6.1 software.

### Fourier Transform Infrared (FT-IR) Spectroscopy

Protein secondary structures of as synthesized silk-sugar lyophilized powders, regenerated silk and reaction intermediates (carboxylated and aminated silk) were determined using a JASCO FTIR 6200 spectrometer (JASCO, Tokyo, Japan) with a MIRacle attenuated total reflectance (ATR) with germanium crystal. FT-IR measurements were performed by averaging 64 scans with a resolution of 4 cm-1 within the wavenumber range of 600 and 4000 cm-1. Data analysis and percentage β-sheet content were performed and calculated using the Fourier self-deconvolution method using Origin software (Origin 2020, OriginLab, Northampton, MA).

### Primary Amine Quantification (TNBSA Assay)

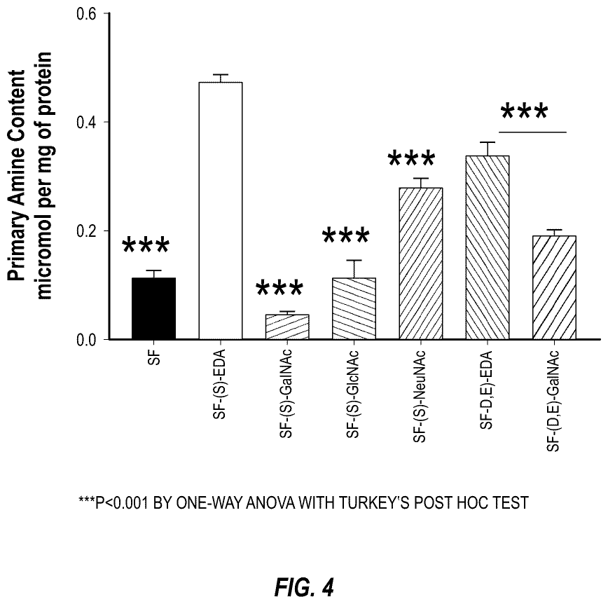

2,4,6-trinitrobenzenesulfonic acid (TNBSA) (Sigma-Aldrich, St. Louis, MO) assay was performed to quantify the total primary amine content of the aminated silk fibroin and different silk-sugar glycopolymers. Silk-sugar glycopolymers and the controls (0.5 mg/ml) were mixed with 0.05% w/v TNBSA solution in 0.4 M NaHCO₃ buffer (pH 8.5) at 1:1 volume ratio. The mixed solutions were incubated for 2 h at 37° C. and its absorption at 420 nm was measured using SpectraMax M2 multimode microplate reader (Molecular Devices, Sunnyvale, CA) (n=5). OD420 values were normalized to dye only and no dye controls and then converted to primary amine content (nmol/mg protein) using the standard calibration curve which was prepared with known concentrations of β-alanine standard solutions. Results are shown in FIG. 4.

### Statistics

ATR-FTIR, biological studies were performed on n=3 independent sample replicates at each condition. Primary amine quantification (TNBSA assay) was performed with n=5 independent samples. All data are expressed as means±standard deviations and used to generate graphical figures. One or two-way analysis of variance (ANOVA) with Tukey's post-hoc multiple comparison test, Bonferroni's test, and unpaired t-test were performed using GraphPad prism (GraphPad Software, San Diego, CA) to determine statistical significance (*p<0.03, **p<0.01, ***p<0.001).

### Results

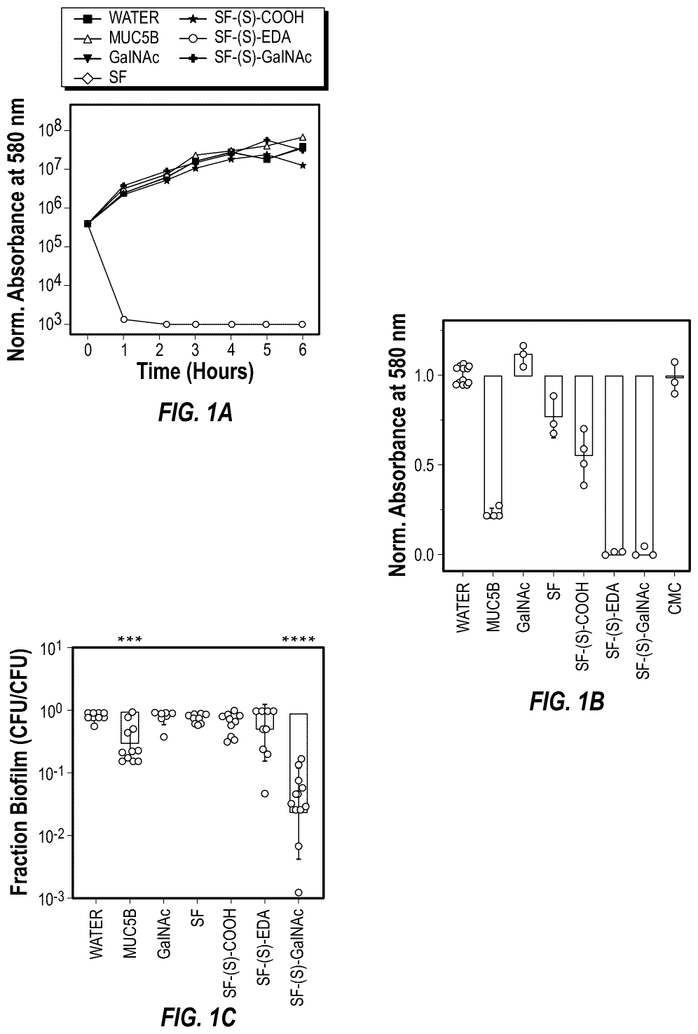

The inventors first tested SF-(S)-GalNAc and its synthetic intermediates to assess whether the silk glycopolymer was cytocompatible for both bacterial and mammalian cells. FIG. 1 illustrates that engineered glycosilk SF-(S)-GalNAc reduces biofilm formation in Streptococcus mutans without killing. Like the mucin MUC5B, silk fibroin and SF-(S)-GalNAc do not affect the overall growth of the bacterial population (see FIG. 1A), but SF-(S)-GalNAc does reduce the number of cells in the biofilm population (see FIG. 1B). The synthetic intermediate SF-(S)-EDA demonstrates antimicrobial activity, likely due to its overall positive charge (see FIG. 1A). MUC5B and SF-(S)-GalNAc reduce the total bacterial biomass, including exopolysaccharides produced by S. mutans (see FIG. 1C). SF-(S)-EDA also reduces biomass because it kills the bacteria. Confocal microscopy of an in vitro culture of S. mutans shows a dense biofilm close to the surface. When treated with SF-(S)-GalNAc or MUC5B, S. mutans bacteria are no longer attached to the surface, while unmodified silk fibroin shows minimal effect. Binding assays show that S. mutans adhere to polystyrene surfaces, but do not strongly bind to MUC5B, Silk Fibroin, and SF-(S)-GalNAc (data not shown, but present in provisional application and can be provided to a patent examiner upon request). Because MUC5B reduces biofilm formation, but silk fibroin does not, this suggests that the primary mechanism by which SF-(S)-GalNAc reduces biofilm formation is not competitive inhibition of bacterial binding to surfaces.

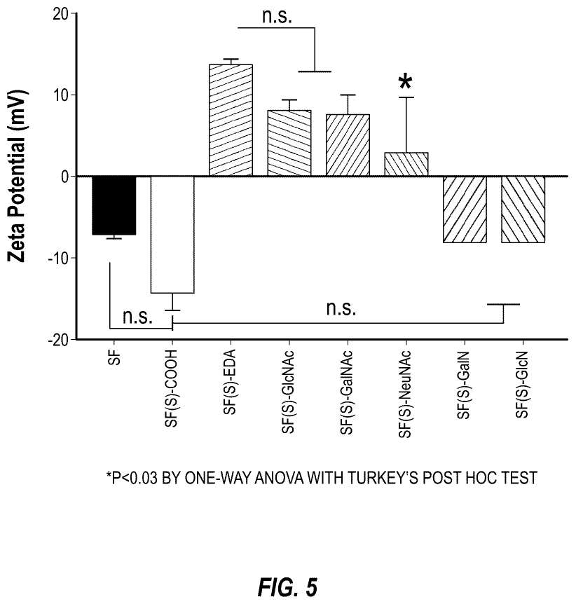

One synthetic intermediate, aminated silk fibroin SF-(S)-EDA was toxic to the bacterial cells, likely because of its high density of amine groups, which could disrupt the bacterial membrane, in a standard antimicrobial polymer mechanism (see FIG. 5). FIG. 5 illustrates the zeta potential (mV) of different protein polymers at 1 wt % in a HEPES buffer (pH 7.4). Zeta potential measures the electrical potential of colloidal dispersions, indicating whether species in solution have a positive or negative charge. Silk fibroin has a negative zeta potential, and SF-(S)-EDA has a positive zeta potential after the reaction of carboxylic acid residues with amines. The zeta potential is lowered as sugar side chains are reacted with the amine groups, but it does not return to a negative zeta potential. These results validate successful carboxylation, amination, and sugar modification steps. Zeta potential is important because it indicates how inherently toxic a polymer solution may be to bacteria: in general, antimicrobial polymers have positive zeta potentials at neutral pH, while most bacteria have negative zeta potential at neutral pH. These positively charged antimicrobial polymers then function by disrupting the bacterial membrane and lysing the cells. This effect was observed in the case of SF-(S)-EDA, which has a high zeta potential and was extremely toxic to S. mutans. The isoelectric point of some Streptococci is under pH 2, indicating that these bacteria have a very negatively-charged surface at neutral pH. (c) FTIR analysis measured absorbance at 1642 cm⁻¹, enabling quantification of [symbol] -sheet conformation in the boiled silk fibroin and its derivatives. Data are presented as mean±standard deviation (n=3). The symbol “*” indicates p<0.03 by one-way ANOVA with Tukey's post hoc test.

In testing mammalian cell cytocompatibility, the present inventors observed that SF-(S)-COOH was toxic, but silk fibroin and other constructs were largely cytocompatible (data supporting these conclusions is omitted here but is present in the provisional application and can be provided to a patent examiner upon request). Additionally, at the effective concentration, 4.5 wt %, silk fibroin and the derivative constructs have a viscosity of 2-6 mPa.s. at a 90 s⁻¹ shear rate, which is similar to the viscosity of healthy saliva, at 3-5 mPa.s. at the same shear rate. The viscosity of polymers at 4.5 wt % solution in water was measured with a rheometer. At this concentration, silk fibroin and the derivative constructs have a viscosity of 2-6 mPa.s. at a 90 s⁻¹ shear rate, which is similar to the viscosity of healthy saliva, at 3-5 mPa.s. at the same shear rate. This similarity holds at lower shear rates as well, where saliva, silk, and silk glycopolymers have a shear rate of 50-100 mPa.s. at 1 s⁻¹. Data for these observations are not included here but are present in the provisional application and can be provided to a patent examiner upon request.

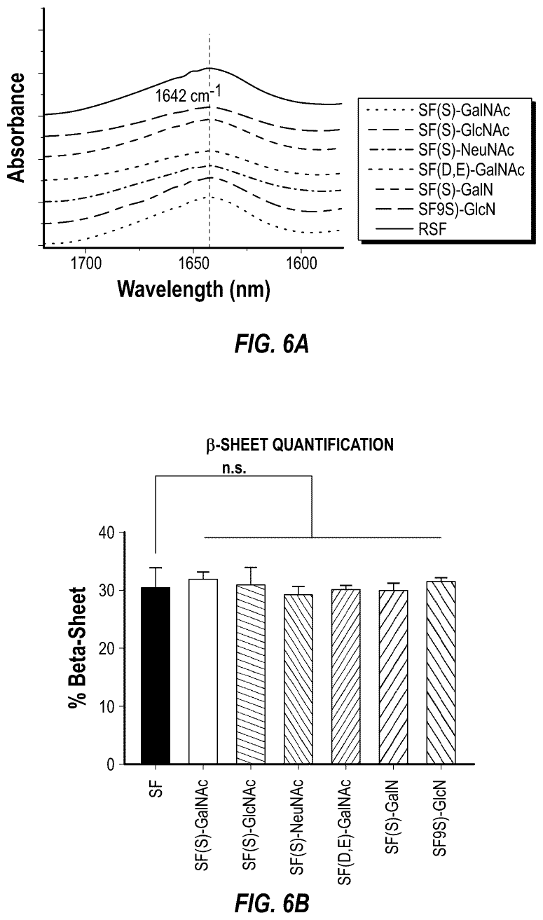

This similarity holds at lower shear rates as well, where saliva, silk, and silk glycopolymers have a shear rate of 50-100 mPa.s. at 1 s⁻¹. Finally, the present inventors assessed how glycan functionalization might alter the structure of the boiled silk fibroin, which was comprised of approximately 30% [symbol] -sheet secondary structures, with the rest of the polypeptide as random coils. Through FTIR, the present inventors found that sugar functionalization did not result in any significant change in polypeptide structure. See FIG. 6.

FIG. 6 illustrates Fourier transform Infra-red (FT-IR) studies of all tested silk-sugar glycopolymers and beta-sheet quantification. FIG. 6A illustrates FT-IR spectra of regenerated SF (Black line 7 in key, line 1 in graph) and sugar modified silk polymers (SF(S)-GalNAc (orange line 1 in key, line 7 in graph), SF(S)-GlcNAc (Dark blue line 2 in key, 6 in graph), SF(S)—NeuNAc (purple line 3 in key, 5 in graph), SF(S)-GalN (light orange line 4 in key, line 4 in graph), SF(S)-GlcN (light blue line 6 in key, line 2 in graph), SF(D, E)-GalNAc (yellow line 5 in key, line 3 in graph). The line numbers in the description here of FIG. 6A refer to the number from the top of the key or graph, respectively, such that “line 1” refers to a topmost line, and “line 7” refers to a bottommost line. All glycopolymers and regenerated silk fibroin show FT-IR absorption peak at 1642 cm⁻¹ corresponding to random coil conformation. FIG. 6B illustrates the percentage Beta-sheet content of SF and SF-sugar glycopolymers prepared from regenerated SF through different chemical modification routes. The results show no significant change in % beta-sheet content between SF and SF-sugar glycopolymers. (Data are presented as mean±standard deviation (n=3). One-way ANOVA with Bonferroni's multiple comparison test was used.)

Then, in order to test whether SF-(S)-GalNAc could reproduce some of mucin's bioactivity, the present inventors tested its ability to prevent S. mutans biofilm formation in a 96-well plate assay (see FIG. 1B). In biofilm-promoting media conditions, 90% of the cells are attached to the surface, and 10% are suspended in culture. When SF-(S)-GalNAc is added to the culture at 4.5%, only 2% of the cells are in a biofilm, with the remaining 98% left suspended. By comparison, cultures with 0.5% MUC5B mucin are 30% biofilm. Soluble GalNAc, silk, and the synthetic silk intermediates do not have a statistically significant impact on fractional biofilm formation.

It is essential to note that SF-(S)-GalNAc are not reducing biofilm by killing the bacteria and reducing the total size of the population. Rather, SF-(S)-GalNAc and MUC5B mucin discourage bacteria from attaching to the surface of the plate, so that they stay suspended in culture. To visualize this shift in community structure, the present inventors stained S. mutans with SYTO9 and performed confocal microscopy (images not shown but are present in the provisional patent application and can be provided to a patent examiner upon request). Normally, bacteria are tightly adhered to the plate surface, but when treated with SF-(S)-GalNAc or mucin, they no longer attach to the surface. Cultures treated with silk did not show a change in community structure.

It is possible that mucin and SF-(S)-GalNAc are preventing biofilm formation by shielding the bacteria from initially binding to surfaces. To test this, the present inventors coated surfaces with mucin, silk fibroin, and SF-(S)-GalNAc, and tested for bacterial adhesion to the polymer coating. As reported previously, S. mutans did not bind to MUC5B mucin. Interestingly, silk-coated surfaces were equally effective in repelling initial S. mutans adsorption, even though they did not reduce biofilm formation, indicating that reducing binding is not sufficient to prevent biofilm formation. Further, S. mutans largely did not bind to SF-(S)-GalNAc coated surfaces, although they did bind to SF-(S)-GalNAc more than unmodified silk fibroin. Data for these observations was included in the provisional application and can be provided to a patent examiner upon request. Together, this suggests that SF-(S)-GalNAc is not preventing biofilm formation solely by acting as a physical barrier.

Given that SF-(S)-GalNAc does not act as a physical barrier, the present inventors hypothesize that it may disrupt biofilm maturation. After initial surface attachment, S. mutans breaks down sucrose to synthesize the exopolysaccharides (EPS) dextran and glucan, embedding the bacteria in an extracellular matrix and forming a mature biofilm. The EPS matrix makes the bacterial biofilm harder to remove, shields microbes from antibiotic treatment, and is associated with increased virulence. Using crystal violet, the present inventors stained S. mutans biofilms and quantified the total biomass, which includes bacterial cells and EPS (see FIG. 1C). SF-(S)-EDA showed a marked decrease in bacterial biomass, which was proportional to its overall reduction of bacterial growth. The mucin MUC5B reduced biomass by 77%. While silk fibroin showed a moderate 24% reduction, SF-(S)-GalNAc reduced biofilm biomass by 98%. This indicates that the presence of SF-(S)-GalNAc induces the bacteria to produce fewer exopolysaccharides.

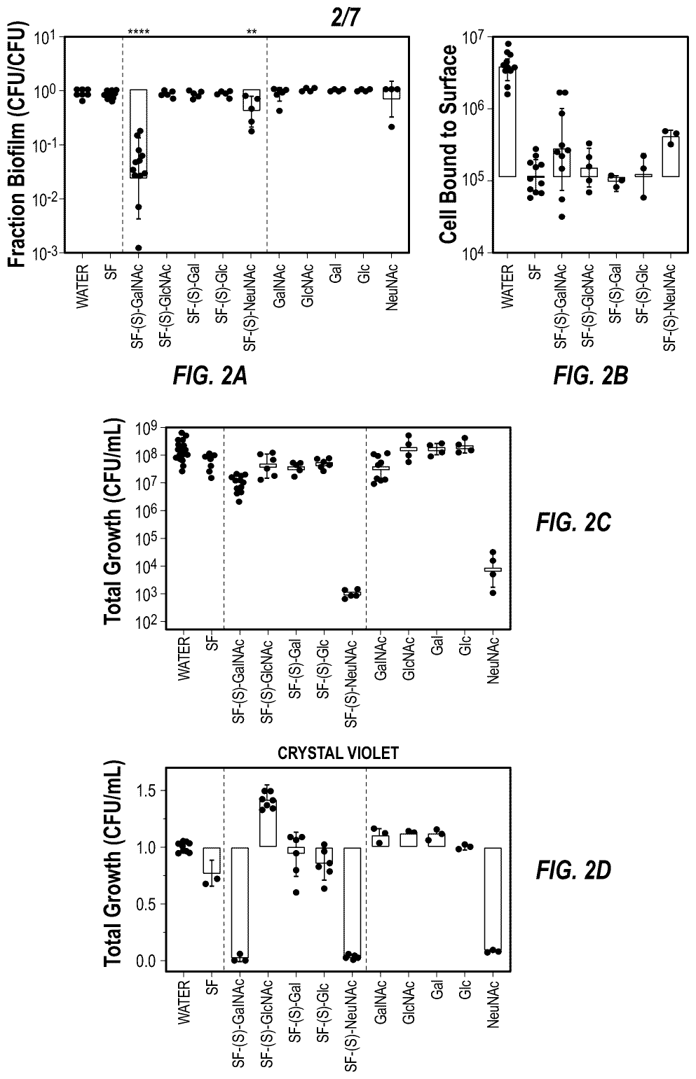

Next, to test whether the effect is unique to GalNAc, the present inventors synthesized a library of silk glycopolymers displaying other monosaccharides found in mucin, specifically GlcNAc, Gal, Glc, and NeuNAc. With regard to FIG. 2, the biofilm reducing capacities of glycosilk is specific to GalNAc grafting. A library of polymers was synthesized with similar sugars, GlcNAc, Gal, Glc, and NeuNAc. Referring to FIG. 2A, only SF-(S)-GalNAc was able to reduce S. mutans cells in the biofilm relative to the whole cell population. Referring to FIG. 2B, S. mutans did not bind to any of the glycopolymers when they were coated on the surface of polystyrene plates. FIG. 2C illustrates that most glyco silk polymers and soluble glycans do not affect the growth of S. mutans. However, SF-(S)-NeuNAc and soluble NeuNAc are toxic to S. mutans. FIG. 2D illustrates that the amount of extracellular polysaccharides were measured using crystal violet staining. SF-(S)-GalNAc treatment significantly reduced the amount of polysaccharides synthesized by S. mutans, indicating that it altered microbial behavior. Conversely, SF-(S)-GlcNAc showed an increase in polysaccharide production, likely because GlcNAc is a building block in peptidoglycan. SF-(S)-NeuNAc also reduced the total polysaccharides produced, likely because it is toxic to S. mutans.

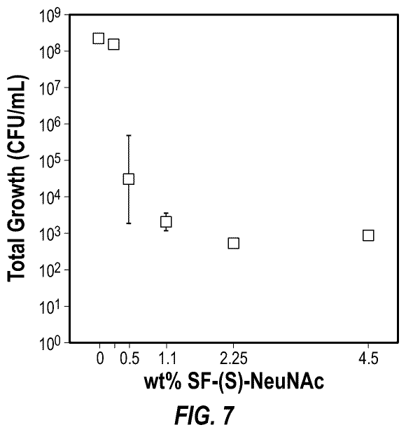

All of these polymers were cytocompatible. SF-(S)-NeuNAc and soluble NeuNAc showed a strong killing effect (see FIG. 2C and FIG. 7), which translated into a reduction in total bacterial biomass as measured by crystal violet (see FIG. 2D), and a small proportional reduction in the fraction of biofilm formed by surviving cells (see FIG. 2A). FIG. 7 illustrates that SF-(S)-NeuNAc is toxic to S. mutans in a concentration dependent manner. On the other hand, SF-(S)-GlcNAc did not affect total growth, and induced an increase in bacterial biomass, potentially because GlcNAc is converted into peptidoglycan in S. mutans's thick cell wall. The other polymers, SF-(S)-Gal and SF-(S)-Glc, did not induce any changes in S. mutans phenotypic behavior. Additionally, S. mutans did not appear to bind to any of the polymers when coated on a well (see FIG. 2B). Together, these results demonstrate that sugar identity is essential for recreating mucin's bioactivity, and that grafted GalNAc has a unique ability to reprogram S. mutans behavior.

Given the specificity of grafted GalNAc's ability to reduce biofilm formation, the present inventors next wanted to assay how grafting density affected efficacy. To achieve this, the present inventors targeted aspartic acid and glutamic acid, which combined represent about 1% of the residues on silk fibroin. the present inventors then directly added an ethylene diamine linker and GalNAc with a carboxybutyl linker, resulting in SF-(D,E)-GalNAc, a polymer with about one-tenth the grafting density of SF-(S)-GalNAc.

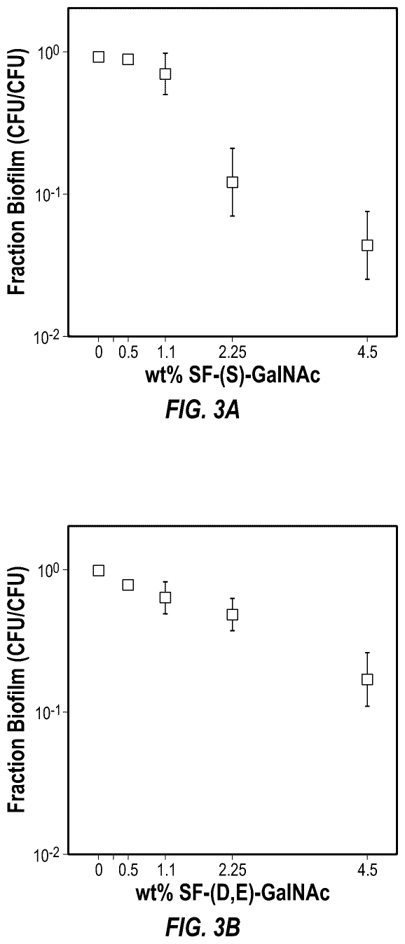

With regard to FIG. 3, biofilm reduction depends on concentration and grafting density of GalNAc on silk fibroin biopolymer. GalNAc (GalNAc-C4-COOH) was covalently conjugated to silk fibroin directly by leveraging the already present carboxylic acid residues (due to Aspartic (Asp) and Glutamic acid (Glu)), which together constitute about 1.1 mol % of the total amino acids present on silk chain, resulting SF-(D,E)-GalNAc. The first step involves amination of the acid content by carbodiimide coupling of ethylene diamine (EDA) in presence of EDC and NHS at pH 6 in 0.05M MES buffer. In the next step, the aminated SF is carbodiimide coupled with acid residues of GalNAc derivative to obtain GalNAc modified SF (SF(D, E)-GalNAc) in a similar reaction environment as in step 1. This results in a lower grafting density compared to SF-(S)-GalNAc polymers. These polymers are not toxic to S. mutans. FIG. 3A illustrates that the biofilm-reducing effect of SF-(S)-GalNAc is concentration-dependent. With higher polymer concentration, higher biofilm-reduction effect was observed. FIG. 3B illustrates that the lower GalNAc-density (SF-(D,E)-GalNAc) polymer also reduces biofilm formation, albeit less effective in comparison to SF-(S)-GalNAc polymer.

This polymer was cytocompatible and did not kill S. mutans. Even with the lower grafting density, SF-(D,E)-GalNAc was still highly effective at reducing fractional biofilm formation (see FIG. 3B). Further, SF-(S)-GalNAc and SF-(D,E)-GalNAc both reduced biofilm formation in a concentration dependent manner (see FIG. 3A). At the highest weight percentage of polymer, SF-(D,E)-GalNAc reduced biofilm formation by 83%, compared to 96% reduction by SF-(S)-GalNAc at the same concentration, indicating that higher grafting density leads to increased potency, albeit in a non-linear manner.

Bacteria are highly attuned to their environments and are capable of quickly altering their phenotypic states in response to specific stimuli. Here, the present inventors saw that S. mutans biofilm formation was reduced by treatment with grafted GalNAc, but that grafted GlcNAc, which only differs by stereochemistry of the C4 position, did not induce any effect. This demonstrates the targeted specificity of the polymer's bioactivity based on which sugar motif is presented. the present inventors expect that similar bioactivity could be achieved by grafting mucin sugars onto other peptide or synthetic polymer backbones, given that the functionality is driven by the presence of GalNAc. However, silk has many advantages as a material backbone: it is non-toxic to human cells, biodegradable, and contains a diverse set of amino acid residues that can be functionalized with further chemistry to introduce orthogonal bioactivity or alter its physical properties.

Beyond increasing the apparent efficacy, grafting glycans onto a material backbone enables the creation of coatings, increases retention time upon application, and presents the opportunity for further functionality, such as lubrication. For instance, the silk solutions have the same viscoelasticity as healthy saliva, enabling them to reproduce mucus's hydrating and lubricating effects. While saliva is a mucosal substance with relatively low viscosity, silk fibroin can be further crosslinked to create a thicker gel that reproduces the density found in thicker mucosal environments, like the gut. Additionally, the specific binding affinity demonstrated by S. mutans towards SF-GalNAc suggests the potential for the creation of coatings that specifically bind and recruit the colonization of beneficial bacteria.

Here, the present inventors created a library of silk-based mucin-inspired glycopolymers that each display specific bioactivity, presenting a platform that could be leveraged to reproduce mucin's protective effects throughout the body. These glycopolymers present a novel strategy for taming microbial infections, as SF-GalNAc functions by preventing the virulent behavior of these bacteria, rather than by killing them. Several opportunistic pathogens have demonstrated unique reductions in virulence properties in the presence of mucin glycans, suggesting that silk glycopolymers could be generated that disarm the virulence of many clinically relevant microbes. This alternative approach to treating bacterial infections draws on the strengths of natural mucosal protective barriers, by enabling the re-domestication of this opportunistic pathogen without detrimentally affecting commensal organisms.

Equivalents and Scope. The recitation of a listing of elements in any definition of a variable herein includes definitions of that variable as any single element or combinations (or sub-combinations) of listed elements. The recitation of an embodiment herein includes that embodiment as any single embodiment or in combination with any other embodiments or portions thereof. Those skilled in the art will recognize or be able to ascertain using no more than routine experimentation, many equivalents to the specific embodiments of the invention described herein. The scope of the present invention is not intended to be limited to the above Description, but rather is as set forth in the following claims:

1. A liquid composition having adequate biofilm-forming microbial population to support formation of a biofilm but having suppressed formation of the biofilm, the liquid composition comprising:
water;
a population of biofilm-forming microbes including optionally bacteria or yeast; and
an O-glycan-substituted silk fibroin having a predetermined degree and distribution of O-glycan substitution, wherein the O-glycan-substituted silk fibroin is not cytotoxic to the population of biofilm-forming microbes,
wherein a comparison liquid composition that lacks the O-glycan-substituted silk fibroin but is otherwise identical to the liquid composition exhibits a comparison degree of formation of the biofilm under biofilm-promoting conditions,
wherein the liquid composition exhibits a degree of formation of the biofilm under the biofilm-promoting conditions,
wherein the degree of formation of the biofilm is less than the comparison degree of formation of the biofilm.

2. The liquid composition of claim 1, wherein the liquid composition has a viscosity of at least 2 millipascal-seconds and at most 6 millipascal-seconds at a 90 s⁻¹ shear rate.

3. The liquid composition of claim 1, wherein the liquid composition has a viscosity of at least 50 millipascal-seconds and at most 100 millipascal-seconds at a 1 s⁻¹ shear rate.

4. The liquid composition of claim 1, wherein the O-glycan-substituted silk fibroin is present in the liquid composition in an amount by weight of between 0.5% and 30%.

5. The liquid composition of claim 1, wherein the liquid composition is free of organic solvents.

6. A biofilm-resistant coating comprising an O-glycan-substituted silk fibroin having a degree of O-glycan substitution, wherein the O-glycan-substituted silk fibroin is not cytotoxic to a population of biofilm-forming microbes including optionally bacteria or yeast,
wherein contacting the biofilm-resistant coating with a microbial liquid composition comprising water and the population of biofilm-forming microbes causes a degree of formation of a biofilm under biofilm-promoting conditions,
wherein contacting a polystyrene surface with the microbial liquid composition causes a polystyrene-comparison degree of formation of a biofilm under biofilm-promoting conditions,
wherein the degree of formation of the biofilm is less than the polystyrene-comparison degree of formation of the biofilm.

7. The liquid composition of claim 1, wherein the degree of formation is reduced by at least 50% relative to the comparison degree of formation.

8. The liquid composition of claim 1, wherein the population of biofilm-forming microbes at least partly or entirely comprises Streptococcus mutans.

9. The liquid composition of claim 1, wherein the population of biofilm-forming microbes at least partly or entirely comprises Staphylococcus aureus, Streptococcus sanguinis, or a combination thereof.

10. A method of making an O-glycan-substituted silk fibroin, the method comprising:
selecting reaction conditions that are intended to provide the O-glycan-substituted silk fibroin with a predetermined degree and distribution of O-glycan substitution, wherein the predetermined degree and distribution of O-glycan substitution exhibits a degree of formation of a biofilm under biofilm-promoting conditions; and
covalently modifying silk fibroin to contain a plurality of O-glycan substitutions using the reaction conditions, thereby providing the O-glycan-substituted silk fibroin.

11. (canceled)

12. (canceled)

13. The liquid composition of claim 1, wherein a mucin-comparison liquid composition that lacks the O-glycan-substituted silk fibroin, contains 0.5% mucin, and is otherwise identical to the liquid composition exhibits a mucin-comparison degree of formation of the biofilm under the biofilm-promoting conditions, wherein the degree of formation of the biofilm is less than the mucin-comparison degree of formation of the biofilm.

14. The liquid composition of claim 1, wherein a glucosamine-comparison liquid composition that lacks the O-glycan-substituted silk fibroin, contains an N-acetyl glucosamine substituted silk fibroin, and is otherwise identical to the liquid composition exhibits a glucosamine-comparison degree of formation of the biofilm under the biofilm-promoting conditions, wherein the degree of formation of the biofilm is less than the glucosamine-comparison degree of formation of the biofilm.

15. The liquid composition of claim 1, wherein a galactose-comparison liquid composition that lacks the O-glycan-substituted silk fibroin, contains galactose-substituted silk fibroin, and is otherwise identical to the liquid composition exhibits a galactose-comparison degree of formation of the biofilm under the biofilm-promoting conditions, wherein the degree of formation of the biofilm is less than the galactose-comparison degree of formation of the biofilm.

16. The liquid composition of claim 1, wherein a glucose-comparison liquid composition that lacks the O-glycan-substituted silk fibroin, contains glucose-substituted silk fibroin, and is otherwise identical to the liquid composition exhibits a glucose-comparison degree of formation of the biofilm under the biofilm-promoting conditions, wherein the degree of formation of the biofilm is less than the glucose-comparison degree of formation of the biofilm.

17. The liquid composition of claim 1, wherein at least a portion of substituted O-glycans are each covalently bound via a serine residue or a threonine residue or wherein each of the substituted O-glycans are covalently bound via the serine residue or the threonine residue.

18. The liquid composition of claim 17, wherein the substituted O-glycans are bound via a carboxyalkyl linker.

19. The liquid composition of claim 18, wherein the carboxyalkyl linker is a carboxybutyl linker.

20. The liquid composition of claim 1, wherein the O-glycan-substituted silk fibroin comprises O-glycan substitutions on between 0.5% and 40.0% of amino acid residues in the O-glycan-substituted silk fibroin.

21. The liquid composition of claim 1, wherein the O-glycan-substituted silk fibroin is at least partly substituted by O-linked N-acetyl galactosamine (GalNAc).

22. The liquid composition of claim 1, wherein the population of biofilm-forming microbes is a population of biofilm-forming bacteria.

23. A method of treating a subject to reduce oral biofilm formation, the method comprising:
administering to the subject's oral cavity an artificial saliva, whereby the artificial saliva combines with contents of the subject's oral cavity to provide either: the liquid composition of claim 1.
